(19) United States
(12) Patent Application Publication
(10) Pub. No.: US 2015/0038895 A1
(43) Pub. Date: Feb. 5, 2015

(54) BOWED TIP FOR LAPAROSCOPIC SURGERY

(71) Applicant: Michael Esposito, Franklin Lakes, NJ (US)

(72) Inventor: Michael Esposito, Franklin Lakes, NJ (US)

(21) Appl. No.: 14/143,076

(22) Filed: Dec. 30, 2013

Related U.S. Application Data
(63) Continuation of application No. 13/954,482, filed on Jul. 30, 2013.

(51) Int. Cl. A61M 1/00; B29D 23/00

(52) U.S. Cl. CPC A61M 1/0084; B29D 23/00
USPC 604/28; 604/43; 264/285

(57) ABSTRACT

An irrigation and suction tip for laparoscopic surgery having an increased range of transport within a surgical cavity during laparoscopic surgery. The tip moves in a circumferential trajectory rather than linearly when a surgeon is irrigating and suctioning tissue. The tip is substantially resilient, withstanding great force and pressure during irrigation and suction procedures. The tip is bowed, having a pair of straight portions, a short distal portion and a long proximal portion joined by a bowed portion, the bowed portion allowing the distal portion to rotate, circumscribing a large area. The bow is limited so that the tip moves within a narrow wall of a cannula of a trocar.

### CROSS-REFERENCE TO RELATED APPLICATIONS

This application is a continuation application of the nonprovisional utility application Ser. No. 13/954,482 filed in the United States Patent Office on Jul. 30, 2013, and claims the priority thereof and is expressly incorporated herein by reference in its entirety, and which is the nonprovisional application of the provisional patent application, Ser. No. 61/682,536 filed in the United States Patent Office on Aug. 13, 2012 and claims the priority thereof and is expressly incorporated herein by reference in its entirety.

### TECHNICAL FIELD

The present disclosure relates generally to a laparoscopic surgical tool. More particularly, the present disclosure relates to a bowed suction and irrigation tip for laparoscopic surgery, including robotic surgery.

### BACKGROUND

Laparoscopic surgery is a modern surgical technique in which a surgeon performs operations in the abdomen of a patient through relatively small incisions (usually 0.5-1.5 cm). Laparoscopic surgery includes operations within the abdominal or pelvic cavities. Older surgical techniques, such as laparotomy, required large abdomen incisions. The laparoscopic procedure is referred to as minimally invasive surgery (MIS) because or the small incisions.

The key element in laparoscopic surgery is the use of a laparoscope which is inserted into the abdomen through a small incision. The abdomen is usually insufflated, or essentially blown up like a balloon, with carbon dioxide gas. This elevates the abdominal wall above the internal organs like a dome to create a working and viewing space. Carbon dioxide is used because it is common to the human body and can be absorbed by tissue and removed by the respiratory system.

There are a number of advantages to the patient with laparoscopic surgery versus an open laparotomy procedure. These include reduced hemorrhaging, reducing the chance of needing a blood transfusion, reduced exposure of internal organs to possible external contaminants thereby reducing the risk of acquiring infections, smaller incisions, reducing pain thereby requiring less pain medication, less post-operative scarring, shorter hospital stay, shorter recovery time with a faster return to everyday living.

While there are many advantages to the patient, laparoscopic surgery requires great surgical skill to offset some of the technical disadvantages of the procedure. The surgeon has limited range of motion at the surgical site resulting in a loss of dexterity and must use tools to interact with tissue rather than directly manipulating by hand, reducing tactile sensation and eliminating palpating tumors. The surgeon must compensate for the misleading depth perception and estimate how much force is being applied to tissue. The endpoints of the surgical tools move in the opposite direction to the surgeon's hands due to the pivot point, making laparoscopic surgery a non-intuitive motor skill that is challenging to learn.

Laparoscopic surgery has been greatly enhanced by the development of robotic minimally-invasive surgery. Instead of directly moving the instruments, the surgeon uses computer-assisted techniques to control the instruments.

Many have proposed improvements to laparoscopic surgical equipment such as flexible rods and shafts with distally attached tools inserted through a trocar placed in the incision. Others have proposed articulated devices that rotate to some degree but do not provide an internal fluid transport channel within the device.

While these units may be suitable for the particular purpose employed, or for general endoscopic use, they would not be as suitable for the purposes of the present disclosure as disclosed hereafter.

While these units may be suitable for the particular purpose employed, or for general use, they would not be as suitable for the purposes of the present disclosure as disclosed hereafter.

In the present disclosure, where a document, act or item of knowledge is referred to or discussed, this reference or discussion is not an admission that the document, act or item of knowledge or any combination thereof was at the priority date, publicly available, known to the public, part of common general knowledge or otherwise constitutes prior art under the applicable statutory provisions; or is known to be relevant to an attempt to solve any problem with which the present disclosure is concerned.

While certain aspects of conventional technologies have been discussed to facilitate the present disclosure, no technical aspects are disclaimed and it is contemplated that the claims may encompass one or more of the conventional technical aspects discussed herein.

### BRIEF SUMMARY

It is an aspect of the present disclosure to provide a tool for laparoscopic surgery that increases a range for irrigating and suctioning. Accordingly, an aspect of the present disclosure provides an irrigation and suction tip for laparoscopic surgery having an increased range of irrigating and suctioning within a surgical cavity.

It is another aspect of the present disclosure to provide a tool for laparoscopic surgery that transports beyond a linear trajectory when a surgeon is irrigating and suctioning tissue. Accordingly, an aspect of the present disclosure provides an irrigation and suction tip for laparoscopic surgery that transports in a circumferential trajectory when a surgeon is irrigating and suctioning tissue.

It is a further aspect of the present disclosure to provide a tool for laparoscopic surgery that is able to transport in a circumferential trajectory and withstand great force and pressure. Accordingly, an aspect of the present disclosure provides an irrigation and suction tip for laparoscopic surgery that is substantially resilient, withstanding great force and pressure during irrigation and suction procedures.

It is yet another aspect of the present disclosure to provide a tool for laparoscopic surgery with an increased trajectory of motion. Accordingly, an aspect of the present disclosure provides an irrigation and suction tip for laparoscopic surgery that is bowed, having a pair of straight portions, a short distal portion and a long proximal portion joined by a bowed portion, the bowed portion allowing the distal portion to rotate, circumscribing a large area.

The present disclosure describes an irrigation and suction tip for laparoscopic surgery having an increased range of transport within a surgical cavity during laparoscopic surgery. The tip moves in a circumferential trajectory rather than linearly when a surgeon is irrigating and suctioning tissue. The tip is substantially resilient, withstanding great force and pressure during irrigation and suction procedures. The tip is bowed, having a pair of straight portions, a short distal portion and a long proximal portion joined by a bowed portion, the bowed portion allowing the distal portion to rotate, circumscribing a large area. The curve of the bow portion is limited so that the tip moves within a narrow wall of a cannula of a trocar.

The present disclosure addresses at least one of the foregoing disadvantages. However, it is contemplated that the present disclosure may prove useful in addressing other problems and deficiencies in a number of technical areas. Therefore, the claims should not necessarily be construed as limited to addressing any of the particular problems or deficiencies discussed hereinabove. To the accomplishment of the above, this disclosure may be embodied in the form illustrated in the accompanying drawings. Attention is called to the fact, however, that the drawings are illustrative only. Variations are contemplated as being part of the disclosure.

### BRIEF DESCRIPTION OF THE DRAWINGS

In the drawings, like elements are depicted by like reference numerals.

The drawings are briefly described as follows.

FIG. 1 is a diagrammatic perspective view of a surgical field prepared for a laparoscopic abdominal procedure with a bowed tip inserting into a trocar.

FIG. 2A is a diagrammatic perspective view of the bowed tip inserted into the trocar.

FIG. 2B, similar to FIG. 2A, is a diagrammatic perspective view of the bowed tip inserted into the trocar, showing a full rotation of a distal end of the tip.

FIG. 2C, similar to FIG. 2A, is a diagrammatic perspective view of the bowed tip inserted into the trocar, an angle of the bowed tip substantially close to a maximum curve of the bow.

FIG. 3A is a diagrammatic perspective view of the bowed tip inserting into the trocar, with a portion having a straight portion inside the trocar.

FIG. 3B, similar to FIG. 3A, is a diagrammatic perspective view of the bowed tip inserting into the trocar, with a portion having a bowed portion inside the trocar.

FIG. 3C, similar to FIG. 3B, is a diagrammatic perspective view of the bowed tip fully inserted into the trocar.

The present disclosure now will be described more fully hereinafter with reference to the accompanying drawings, which show various example embodiments. However, the present disclosure may be embodied in many different forms and should not be construed as limited to the example embodiments set forth herein. Rather, these example embodiments are provided so that the present disclosure is thorough, complete and fully conveys the scope of the present disclosure to those skilled in the art.

### DETAILED DESCRIPTION OF THE PREFERRED EMBODIMENTS

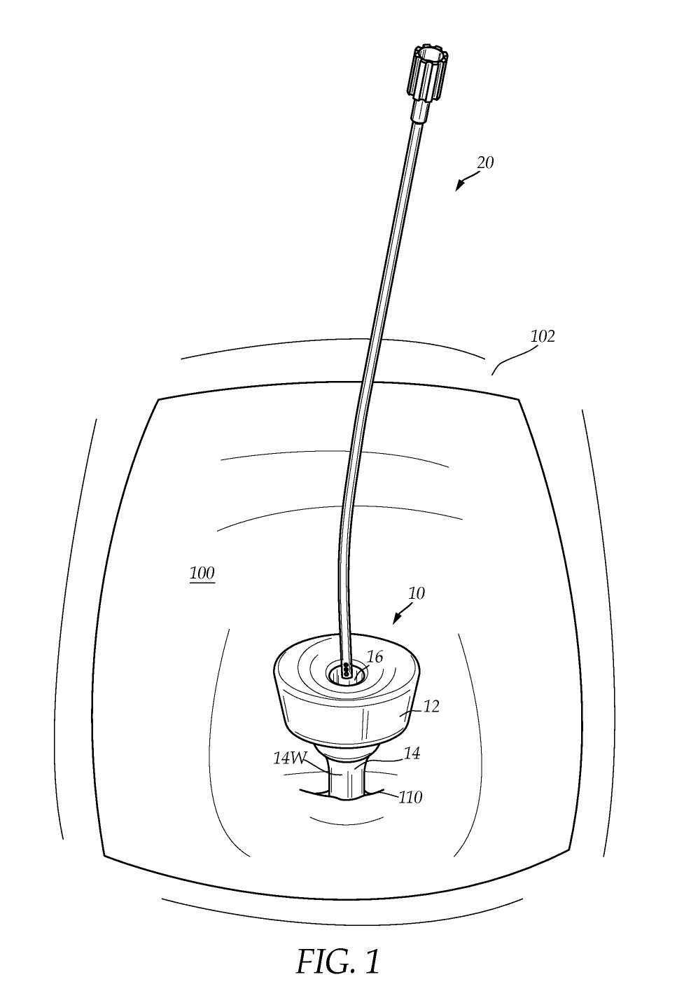

FIG. 1 illustrates a bowed suction and irrigation tip 20 approaching a trocar 10 inserted in a patient's abdomen 100. The illustration shows a laparoscopic procedure in process, with the abdomen 100 typically draped with a plurality of surgical drapes 102, exposing an exterior field of surgery. In this disclosure, the term laparoscopic procedure includes robotic minimally-invasive surgery and other surgical procedures that are performed through a trocar inserted into a torso of a patient and the term laparoscopic procedure is not a limitation.

The trocar has been inserted through a small incision 110, generally about 1 to 2 centimeters in length. The trocar has a collar 12 and a cannula 14, the collar 12 having an opening 16 in fluid communication with the cannula 14. Trocars are well known to those of ordinary skill in the art and the illustration does not show the trocar in detail, but only showing those elements common to many.

While the illustration shows the exterior surgical field, what is not easily illustrated is an interior surgical field that is accessed through the trocar. A user, for example, a surgeon, a surgery technician or other operating room personnel, access the interior surgical field through the trocar 10 by inserting a surgical tool through the opening 16 and into the cannula 14. The user sees the interior surgical field through a variety of devices, such as a camera or fiber optics which are well known to those of ordinary skill.

The opening of the trocar is typically annular and is provided with an interior diameter that accommodates differing instruments. For example, typically the interior diameters of the opening and cannula is 8 mm for robotic instruments, 5 mm for irrigation and suction and 12 mm for larger (non-robotic) surgical instruments, such as a stapler. Often in robotic surgery only one incision is made and irrigation and suction is performed through a single trocar having 8 mm interior diameter cannula. When single trocar surgery is performed, the opening of the trocar and the cannula becomes very crowded with a plurality of instruments.

The cannula 14 of the trocar has a wall 14W which is typically annular in cross-section, having a diameter, the diameter of the wall cross-section matching the diameter of the opening of the trocar to which it is in fluid communication. All surgical tools must fit through the narrow cross-section diameter of the trocar cannula to reach the internal surgical field.

The bowed suction and irrigation tip 20 is about to be inserted into the opening 16. The tip 20 must fit within the opening 16 and cannula 14, the wall 14W of the cannula being substantially rigid. The tip 20 is used in suctioning tissue and fluids with a vacuum as well as irrigating the internal surgical field by delivering a fluid, thereby the tip being substantially resilient, able to withstand a force from the fluid delivery and a force of the vacuum required to suction tissue and fluids.

In one example embodiment, the tip 20 is provided in a reusable form, constructed from materials that are capable of repeated sterilization, such as metal and engineered plastics. For example, the tip is constructed from, but not limited to, stainless steel, titanium, titanium steel, titanium-based alloys, nickel-based alloys, superalloys, zirconium and hafnium alloys and engineered plastics such as for example, but not limited to, polyether ether ketone (PEEK). For the purpose of this disclosure, the term resilient means a material that requires some force or pressure to change shape, without permanent deformation or rupture but easily recovers its shape after a force is applied.

In a further example embodiment, the tip is provided in a disposable form, constructed from engineered and commodity plastics, that are initially sterilized such as, but not limited to PET (polyethylene terephthalate), polyethylene, polycarbonate and copolymers. The disposable bowed tip is substantially pliable, having a bow providing a substantial range of motion when inserted through the narrow cannula as will be explained hereinbelow. For the purpose of this disclosure, the term pliable means a material supple enough to bend freely without breaking.

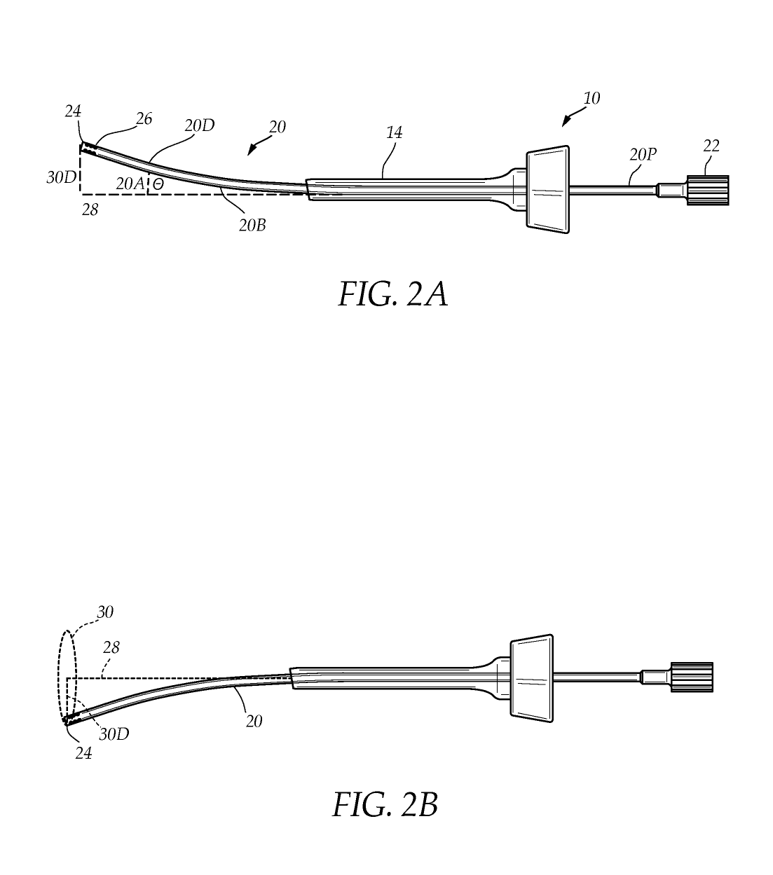

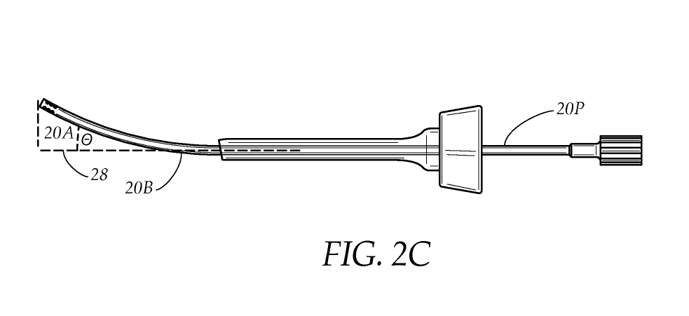

FIG. 2A shows the trocar and the tip free-standing from the surgical field to demonstrate the features of the bowed tip 20. The tip is a hollow tube having a wall, the hollow tube has a length ranging from about 40 cm to 60 cm.

The tip has a straight proximal portion 20P, having a proximal end proximal end exterior to a trocar when the tip is inserted therein, the proximal end attaching to a connector 22 that selectively connects the tip with a suction or irrigation source. The tip has a straight distal portion 20D, the distal portion terminating with a blunt end 24 having a plurality of apertures 26 for an egress of irrigation fluid during an irrigation procedure and an ingress of tissue and fluids during a suction procedure. The blunt end is firm. For the purpose of this disclosure, the term firm means a material that resists pressure and is not easily deformed.

Connecting the proximal portion and the distal portion is a bow portion 20B, the bow portion in fluid communication with the proximal portion 20P and the distal portion 20D, the bow portion producing a curve in the tube, the curve having an angle θ 20A, the angle subtending the bow portion with an imaginary straight reference line 28 of the proximal portion, the curve enabling the blunt distal end 24 of the tube to circumscribe a large area while manipulating the proximal end of the tube without manipulating the trocar, said trocar having said tube inserted therein.

In one embodiment, the blunt end 24 curves away from the imaginary line 28 at a distance slightly more about 5 mm at minimum and about 8 mm at maximum, so that the tip inserts within the 5 mm inner diameter of the cannula 14 within the cannula walls 14W as described hereinbelow.

In one embodiment, the proximal portion, bow portion and distal portion form a unitary piece. In further embodiments the portions are connected to form a unitary piece.

The hollow tube has a midpoint equidistant between the distal end 20D and the proximal end 20P and the bow portion is between said midpoint and said distal end. In one embodiment, the bow portion 20B is positioned somewhat towards the distal blunt end 24, around twenty percent of the length of the tip from the blunt end, the distal portion and the proximal portion having a ratio of around 1:4 respectively.

The proximal portion continues in the imaginary straight line 28, the imaginary straight line a reference line for describing the bowing of the tip. The bow portion 20B forms an angle θ 20A of a range of about 5 to 30 degrees, preferably 15 to 25 degrees with the straight line 28 of the proximal portion 20P. In FIG. 2A, the angle θ 20A is about twenty degrees.

FIG. 2C illustrates a further example embodiment, the bow portion 20B forming the angle θ 20A at a maximum curvature of 30 degrees with the straight line 28 of the proximal portion. In this example embodiment, the tip 20 is formed from pliable material.

FIG. 2B clearly illustrates the advantages of the bowed tip 20. Unlike straight tips that are well-know in the prior art which only suction or irrigate at a single point within the interior surgical field, the user having to manipulate the trocar from the exterior surgical field within the incision, possibly causing the incision to expand to move the blunt end of the straight tip within the field, the bowed tip circumscribes a larger area within the interior surgical field without manipulating the trocar from the exterior surgical field. Within the larger area now available to the user, the user can irrigate and suction behind a plurality of organs and tissue masses with a minimum of disturbance with potential for damage to said organs and masses.

As demonstrated in FIG. 2B, the blunt end 24 of the tip 20 circumscribes a circular area 30 having a radius 30D of at least 5 mm and an area of at least 78.5 mm squared, the radius having the length from the blunt end 24 to the imaginary line 28 continuing from the proximal portion. With slight manipulation of the trocar, the range of the blunt end 24 is greatly enhanced beyond the at least 78.5 mm squared, allowing the user greater range when irrigating and suctioning. Further advantageously, the bowed tip eliminates parallax error for the user as the blunt end, the distal portion and the bowed portion are no longer directly in the line of vision.

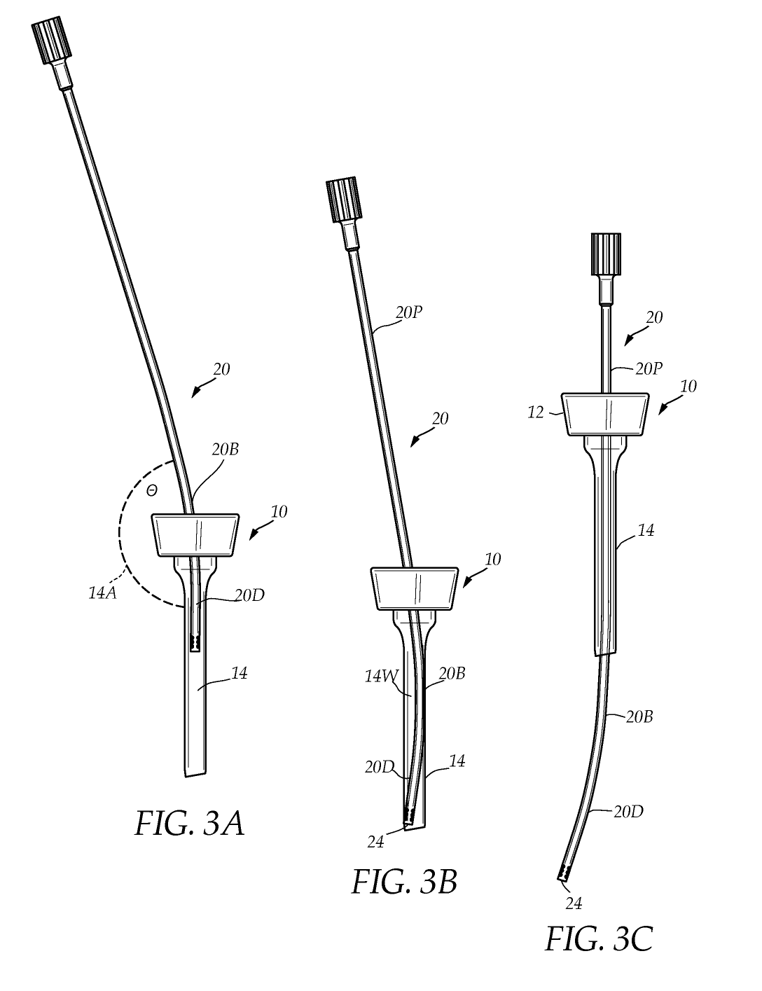

FIGS. 3A, 3B and 3C show the insertion of the tip 20 into the trocar 10 in stages, demonstrating how the bowed tip conforms to the cannula 14. In FIG. 3A, the distal portion 20D inserts into the trocar 10 and down the cannula 14 to the bow portion 20B is in the opening, the proximal portion held at about a 15 to 25 degree angle θ 14A with reference to the cannula, the angle θ equal to the angle θ described hereinabove with reference to the angle of the bow. In FIG. 3B, the tip 20 is slightly tilted while the trocar 10 remains essentially fixed. The blunt end 24 is in contact with the cannula wall 14W, the bow portion 20B is in contact with the cannula wall opposite the blunt end 24. In FIG. 3C, the proximal portion 20P is vertically upright through the trocar collar 12 and cannula 14, the bow portion 20B and the distal portion 20D protruding into interior surgical field. The blunt end freely rotates, circumscribing a larger area in the surgical field.

To use the bowed tip, the user inserts the tip into the opening of the trocar collar, and down the cannula 14 until the bow portion 20B, reaches the opening of the collar, the proximal portion held at about a 15 to 25 degree angle θ 14A with reference to the cannula. The user slightly tilts the tip while the trocar 10 remains essentially fixed, contacting the cannula wall with the blunt end and contacting the bow portion 20B with the cannula wall opposite the blunt end 24. The user uprights the proximal portion 20P through the trocar collar 12 and cannula 14, inserting the bow portion 20B and the distal portion into interior surgical field. The blunt end freely rotates, circumscribing a larger area in the surgical field. The user irrigates or suctions as needed and can access a larger area by gently manipulating the trocar.

To withdraw the tip, the user reverses the steps, by withdrawing the tip until the bowed portion reaches the collar, tilting the tip to the angle θ described hereinabove and further withdrawing the bowed portion and the distal portion from the trocar.

The advantages of the bowed tip 10 are numerous as disclosed hereinabove. The blunt end 24 has a large area for irrigating and suctioning, circumscribed by the end when rotated and is not limited to a single point that limits a straight tip. The large area allows the user to reach around and behind organs and tissue masses that are in the surgical field. The bowed tip can be manipulated by the user with minimal disturbance of the trocar in the incision and the bowed tip eliminates parallax error for the user.

Referring to FIG. 2A, a method of manufacturing a suction and irrigation tip for inserting into a trocar can be demonstrated. The method comprises providing a hollow tube 20, said tube having the distal portion 20D having the blunt distal end 24, said tube having a proximal portion 20P. The plurality of apertures 26 are provided on the wall of said tube adjacent to said blunt distal end 24. The hollow tube is bent, thereby creating a bow portion 20B connecting the distal portion and the proximal portion of the hollow tube, the bow portion in fluid communication with the proximal portion and the distal portion, the bow portion producing a curve in the tip 20, the curve enabling the blunt distal end of the tube to circumscribe a large area when inserted into an internal surgical field through a trocar while manipulating the proximal end of the tube without manipulating said trocar.

Once the tip is bent, it is sterilized in preparation for use in surgical procedures including the disposable form, constructed from engineered and commodity plastics and the reusable form constructed from materials that are capable of repeated sterilization.

It is understood that when an element is referred hereinabove as being “on” another element, it can be directly on the other element or intervening elements may be present therebetween. In contrast, when an element is referred to as being “directly on” another element, there are no intervening elements present.

Moreover, any components or materials can be formed from a same, structurally continuous piece or separately fabricated and connected.

It is further understood that, although ordinal terms, such as, “first,” “second,” “third,” are used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein.

Spatially relative terms, such as “beneath,” “below,” “lower,” “above,” “upper” and the like, are used herein for ease of description to describe one element or feature's relationship to another element(s) or feature(s) as illustrated in the figures. It is understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device can be otherwise oriented (rotated 90 degrees or at other orientations) and the spatially relative descriptors used herein interpreted accordingly.

Example embodiments are described herein with reference to cross section illustrations that are schematic illustrations of idealized embodiments. As such, variations from the shapes of the illustrations as a result, for example, of manufacturing techniques and/or tolerances, are to be expected. Thus, example embodiments described herein should not be construed as limited to the particular shapes of regions as illustrated herein, but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

In conclusion, herein is presented a bowed suction and irrigation tip for laparoscopic surgery. The disclosure is illustrated by example in the drawing figures, and throughout the written description. It should be understood that numerous variations are possible, while adhering to the inventive concept. Such variations are contemplated as being a part of the present disclosure.

1-11. (canceled)

12. A method of manufacturing a suction and irrigation tip for inserting into a trocar, comprising:
providing a hollow tube having a wall, said tube having a distal portion having a blunt distal end, said tube having a proximal portion;
providing a plurality of apertures on the wall of said tube adjacent to said blunt distal end; and
bending said hollow tube, thereby creating a bow portion connecting the distal portion and the proximal portion of the hollow tube, the curve of the bow portion positioned towards the distal end, the curve of the bow limited from five degrees to thirty degrees, the bow portion positioned around twenty percent of the length of the tip from the distal end, the bow portion in fluid communication with the proximal portion and the distal portion, the bow portion producing a curve in the tube, the curve enabling the blunt distal end of the tube to circumscribe a large area when inserted into an internal surgical field through a trocar while manipulating the proximal end of the tube without manipulating said trocar.

13. (canceled)

14. The method of manufacturing the suction and irrigation tip as described in claim 12, wherein the hollow tube is provided in a disposable form, constructed from engineered and commodity plastics selected from the group consisting of polyethylene terephthalate, polyethylene, polycarbonate and copolymers.

15. The method of manufacturing the suction and irrigation tip as described in claim 12, wherein the hollow tube is provided in a reusable form, constructed from materials that are capable of repeated sterilization, such as metal and engineered plastics selected from the group-consisting of stainless steel, titanium, titanium steel, titanium-based alloys, nickel-based alloys, superalloys, zirconium and hafnium alloys and polyether ether ketone and the step of sterilizing the hollow tube is repeated.

16. The method of manufacturing the suction and irrigation tip as described in claim 12, wherein the hollow tube is a unitary piece.

17. A method of using a bowed suction and irrigation tip for inserting into a trocar, comprising:
inserting a suction and irrigation tip having a proximal portion, a distal portion, and a bowed portion, said bowed portion having a curve with an angle, the bowed portion therebetween the proximal portion and distal portion, said distal portion inserting into a trocar, the trocar having a collar with an opening fluidly connecting to a trocar cannula, the cannula having an interior wall, said distal portion of said tip inserting through the opening and into said trocar cannula until the bowed portion reaches the opening, the proximal portion held at an angle, said angle equal to the angle of the curve of said bowed portion;
tilting the proximal portion of said tip while the trocar remains essentially fixed, further inserting said tip as the distal portion is within and contacting the cannula interior wall and the bow portion is within and contacting the cannula interior wall opposite the blunt end;
uprighting the proximal portion through the trocar opening and cannula, further inserting said tip into the trocar, thereby inserting the bow portion and the distal portion into an interior surgical field, the distal end freely rotating, circumscribing a large area in the surgical field, providing greater access for irrigating and suctioning in the surgical field; and
irrigating and suctioning in the surgical field while rotating the tip.

18. The method of using a bowed suction and irrigation tip in a trocar as described in claim 17, wherein the step of irrigating and suctioning in the surgical field while rotating the tip is followed by step of withdrawing said tip until the bowed portion reaches the opening, tilting said tip to said angle and further withdrawing the bowed portion and the distal portion from the trocar.

19. The method of using a bowed suction and irrigation tip in a trocar as described in claim 17, wherein the angle of the curve of the bow portion ranges from five degrees to thirty degrees.
